(19) United States
(12) Patent Application Publication
(10) Pub. No.: US 2018/0256122 A1
(43) Pub. Date: Sep. 13, 2018

(54) DIGITAL RADIOGRAPHIC IMAGE MODULE

(71) Applicant: Carestream Health, Inc., Rochester, NY (US)

(72) Inventors: Timothy J. Wojcik, Rochester, NY (US); Bradley S. Jadrich, North Port, FL (US); Jeffery R. Hawver, Marion, NY (US)

(21) Appl. No.: 15/911,232

(22) Filed: Mar. 5, 2018

Related U.S. Application Data
(60) Provisional application No. 62/467,842, filed on Mar. 7, 2017.

(51) Int. Cl. A61B 6/00

(52) U.S. Cl. CPC A61B 6/4283; A61B 6/541; A61B 2090/3966; A61B 6/4291; A61B 6/547

(57) ABSTRACT

An enclosure for a digital radiography detector includes a handle, a connector configured to electrically and mechanically engage the detector and an accessory cavity to provide storage space for an accessory that is electrically and mechanically engageable to the detector.

### CROSS REFERENCE TO RELATED APPLICATIONS

This application claims the benefit of U.S. Provisional application U.S. Ser. No. 62/467,842, provisionally filed on Mar. 7, 2017, entitled “DIGITAL RADIOGRAPHIC IMAGE MODULE”, in the names of Timothy J. Wojcik, Bradley S. Jadrich, and Jeffery R. Hawver, which is hereby incorporated by reference herein in its entirety.

### TECHNICAL FIELD

The application generally relates to digital x-ray imaging methods and systems, and more specifically, to methods and/or systems that use digital radiography (DR) detectors, and more particularly to a modular accessory package for a portable DR detector.

### BACKGROUND

Portable DR detectors are used with many varied radiographic systems such as in-room mobile radiographic systems, portable radiographic systems, NDT systems, at many varied examination locations, such as in field use, under bedridden patients, and at many varied conditions. Using portable DR detectors, hospitals and other healthcare facilities now have expanded capability for obtaining x-ray images, including images obtained at the patient bedside. Expanded opportunities for radiographic imaging in NDT and security applications, veterinary applications, and outdoor use are now possible using DR detectors, including both tethered and wireless DR detectors.

Among the challenges faced in adapting to a broad range of environments is the need for highly adaptable packaging solutions that provide added capabilities for support of the DR detector in different applications. Not only is it necessary to protect the sensitive DR detector from damage in various environments; additional usability factors must also be considered.

Conventional packaging solutions for x-ray cassettes and electronic sensor components fail to take advantage of lightweight DR detector design and the use of more flexible thin substrates. Thus, it can be appreciated that there is a need for improved modular accessory housing or kits for portable DR detectors.

The discussion above is merely provided for general background information and is not intended to be used as an aid in determining the scope of the claimed subject matter.

### SUMMARY

An object of the present disclosure is to advance the art of digital radiography, with particular attention to DR detector packaging. Embodiments of the present disclosure address the need to provide modular packaging solutions that provide a protective encasement for the DR detector, allow addition of a number of support features, and help to make the DR detector more readily usable in a wide range of applications.

These objects are given only by way of illustrative examples, and such objects may be exemplary of one or more embodiments of the disclosure. Other desirable objectives and advantages inherently achieved may occur or become apparent to those skilled in the art. The invention is defined by the appended claims.

In one embodiment, an enclosure for a standalone DR detector includes a handle, a connector configured to electrically and mechanically engage the DR detector and an accessory cavity to provide storage space for an accessory that is electrically and mechanically engageable to the DR detector.

In one embodiment, there is provided a protective enclosure for a standalone DR detector comprising a shell configured to cover one or more surfaces of the DR detector, a handle for carrying the DR detector, a connector for an external tether, and an accessory cavity providing storage space for one or more accessories.

In one embodiment, a method is disclosed that provides a shell having an interior space configured to receive and enclose a standalone DR detector. The shell includes a cavity to receive an accessory. The DR detector is inserted into the shell and a tether is electrically and mechanically connected to the enclosed DR detector through an opening in the shell. A position locator is placed within the cavity, and is configured to determine a location of the position locator and to transmit its location to a receiver, while the DR detector is used in the normal course.

The summary descriptions above are not meant to describe individual separate embodiments whose elements are not interchangeable. In fact, many of the elements described as related to a particular embodiment can be used together with, and possibly interchanged with, elements of other described embodiments. Many changes and modifications may be made within the scope of the present invention without departing from the spirit thereof, and the invention includes all such modifications. The drawings below are intended to be drawn neither to any precise scale with respect to relative size, angular relationship, relative position, or timing relationship, nor to any combinational relationship with respect to interchangeability, substitution, or representation of a required implementation.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIGS. 1A-1C are perspective diagrams that show aspects of an embodiment of a modular accessory container for a portable DR detector;

FIG. 1D shows modifications to accessory cavity for password protection and other features;

FIG. 2 is a schematic diagram that shows another exemplary embodiment of a modular accessory container for a portable DR detector according to an embodiment of the present application;

FIGS. 3A-3F show a low-cost accessory shell that can provide, for a modular accessory enclosure, a means to attach, encase, or retain an anti-scatter material or a sheet of anti-scatter lead plating to a back of a portable DR detector;

FIG. 4A shows a low-cost accessory shell according to an alternate embodiment;

FIGS. 4B and 4C show exploded views of a protective cast foam shell that can be used with the DR detector;

FIGS. 5 and 6 show top, side, and bottom views of a DR detector that can be used with the modular accessory enclosure according to an embodiment of the present disclosure;

FIG. 7 is a perspective view that shows a DR detector along with a modular accessory enclosure that is optimized for mobile imaging;

FIG. 8 is a perspective view that shows a mobile radiography apparatus that can use the DR detector provided with a modular accessory enclosure according to an embodiment of the present disclosure;

FIG. 9 shows use of a DR detector within a cassette frame accessory;

FIG. 10 shows an image module used with a bucky adapter according to an embodiment of the present disclosure;

FIGS. 11 and 12 show an embodiment using a long-length imaging stand for cassette positioning;

FIG. 13 shows an embodiment showing enhanced DR detector source position orientation detection for improved geometric calibration;

FIG. 14 shows an embodiment of modular accessory enclosure suited for bedside imaging; and

FIG. 15 shows an embodiment of a modular accessory enclosure with respiratory and pulse oximeter monitors.

### DETAILED DESCRIPTION OF THE EMBODIMENTS

Currently, DR detectors that are used in a medical facility compared to DR detectors used in portable or field use are different in several respects, but can be served by a common DR detector configured with optional accessories. Embodiments of an accessory container, housing, shell or enclosure for portable DR detectors can provide a system solution and can provide a set of capabilities using modular accessory attachments. Embodiments of an accessory container for portable DR detectors can include a flexible enclosure that acts as a skin, dependent on the form of the DR detector body for structural shape. The accessory enclosure can be a disposable low cost shell providing environmental protection, such as a fluid tight seal, but with reduced shock protection. The accessory enclosure can have suitable environmental robustness, e.g., conforming to military standard guidelines. Such a container or housing may be used for different x-ray exposure techniques. The embodiments disclosed herein pertain to an accessory shell, case, or housing compatible with a fully operable standalone DR detector that may be typically used in the field without such an accessory shell.

According to one embodiment, the accessory shell, housing or enclosure can be closable or sealable in a fluid-tight fashion. The accessory shell or enclosure may be formed as a modular unit to independently accessorize features for various applications.

A number of radiographic applications require a level of environmental robustness and x-ray exposure technique latitude. For example, field use at temperatures from −40° to 140° F.; proofing from or resistance against water and bodily fluids (medical) and rain (non-destructive testing); ruggedness to withstand drops from 4 feet, such as for military standards; backscatter control from poorly collimated and/or pointed x-ray sources as well as from higher energy beams; accommodation of source-detector alignment instruments; a handle to improve ergonomics; and structure to attach source-detector alignment aids, such as a bucky.

Certain exemplary accessory shell or enclosure embodiments can provide various features, including: a removable handle and removable grid; a removable backscatter shield; alternate radio (space allocation only); a manual only, tool free implementation; a robust non-slip surface; alternate digital image transport (e.g., flash memory, space allocation only; battery access; environmental seal; tube to grid alignment; DR detector location detection; dual energy sync circuitry; extra battery; increased bending resistance; point loading capacity; high speed ports; information and status display; inductive battery charging; and backwards system compatibility.

Accessory shell or enclosure embodiments may include a sealable flexible envelope with bumpers, pockets, and an attached handle that may be used to house power and electronics. The accessory shell or enclosure may include the following characteristics: connects power/communications from enclosed internal DR detector to outside connector, retaining water resistance; accommodates a selection of accessories, such as a snap-on grid, a backscatter shield in a pocket, source alignment instruments, temperature controller with portable flat panel DR detector (FPD) battery and insulated blanket.

Certain exemplary accessory shell or enclosure embodiments can provide high image quality, low cost, light weight, disposability (inexpensive vacuum formed plastic shell), scratch and abrasion protection, surface treatment to increase gripping friction, drop-shock and weight bearing durability, fast image access and cycle time, fast availability from cold start, long battery charge life, reliable operation, fast and easy movement/registration between various host systems, support of dual energy and tomosynthesis, abuse sensing and indicating components. Non-bucky imaging features may include source to DR detector alignment aids, fluid and particle tolerance, high backscatter tolerance due to poor exposure technique/alignment, attitude sensing, location tracking, and roaming, proprietary point-to-point connectivity option.

In certain exemplary embodiments, a portable DR detector accessory kit, shell, enclosure, or sleeve can provide continued compatibility with commercially available film/CR cassette accessory devices such as slip on grids, protective bags, positioning stands/holders, and protective shells for high weight bearing exams. In addition, a snap-on handle that is light weight, with cavities to accommodate additional Power over Ethernet connected radio such as UWB or alternative non-802.11 for mobile; optional USB or PoE powered accessories such as pulse oximeter, position locators, audio alarms for unintended out-of-range transport or anti-theft warning. (USB provided from PoE to USB converter). New accessory shell with handle that accepts the various sized cassettes, has high fluid and particle resistance, and allows several options, such as mechanical features that interface with position holding devices (orthopedic, veterinary, cross-table stands, kick-stand and robot grippers for NDT-security); and reduced power consumption, local radio such as ZigBee or IEEE 802.15.4 for wake-up.

Additional exemplary accessory shell or enclosure embodiments can provide a magnetic tether connector running Power over Ethernet, passes the tether connection to second connector outside of the shell housing to enable interface with other Ethernet or charge circuit connections. An alternative construction could include thermal insulation, a temperature controller and material for extreme temperature tolerance.

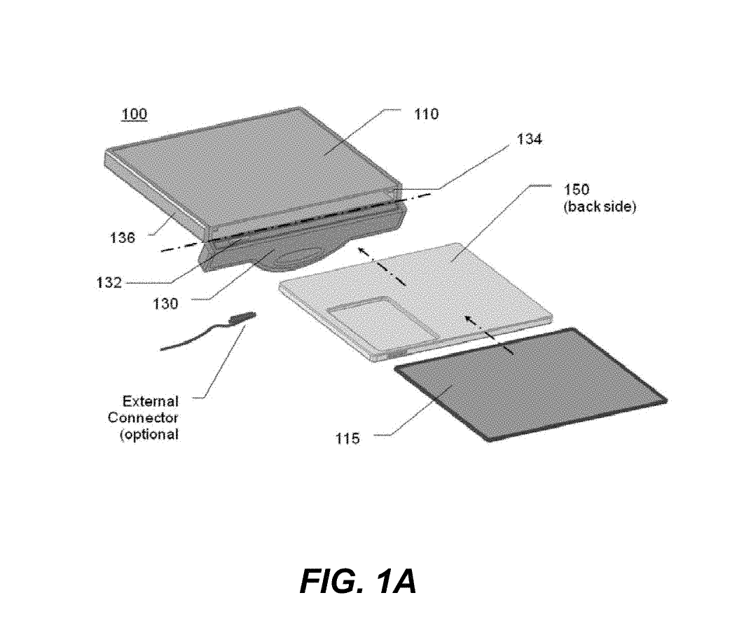

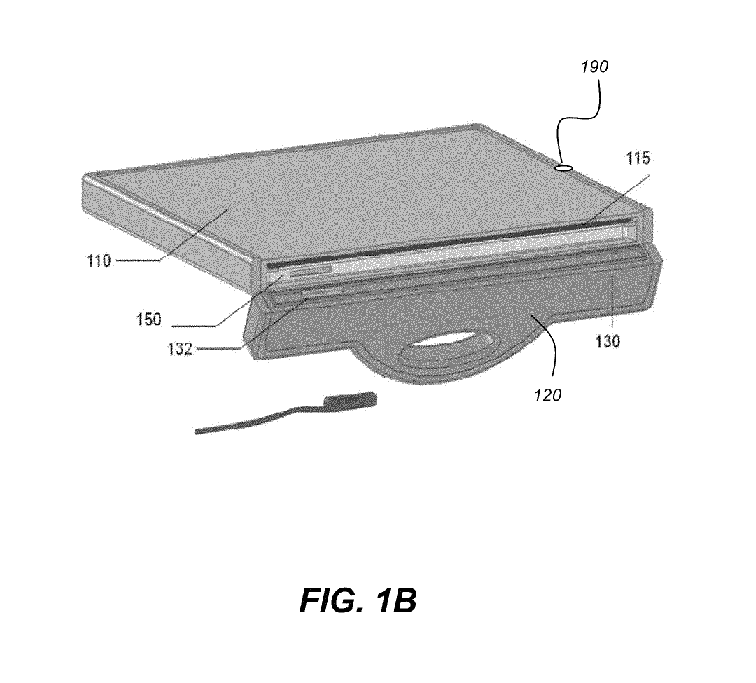

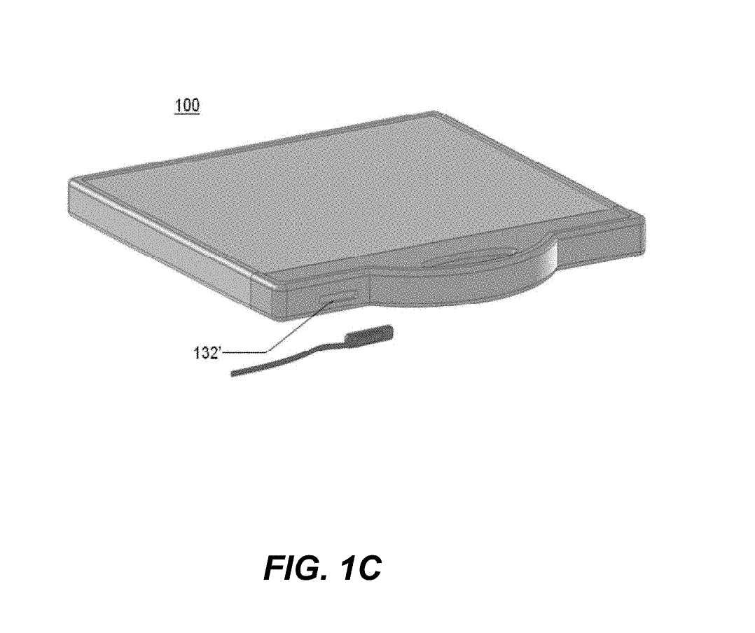

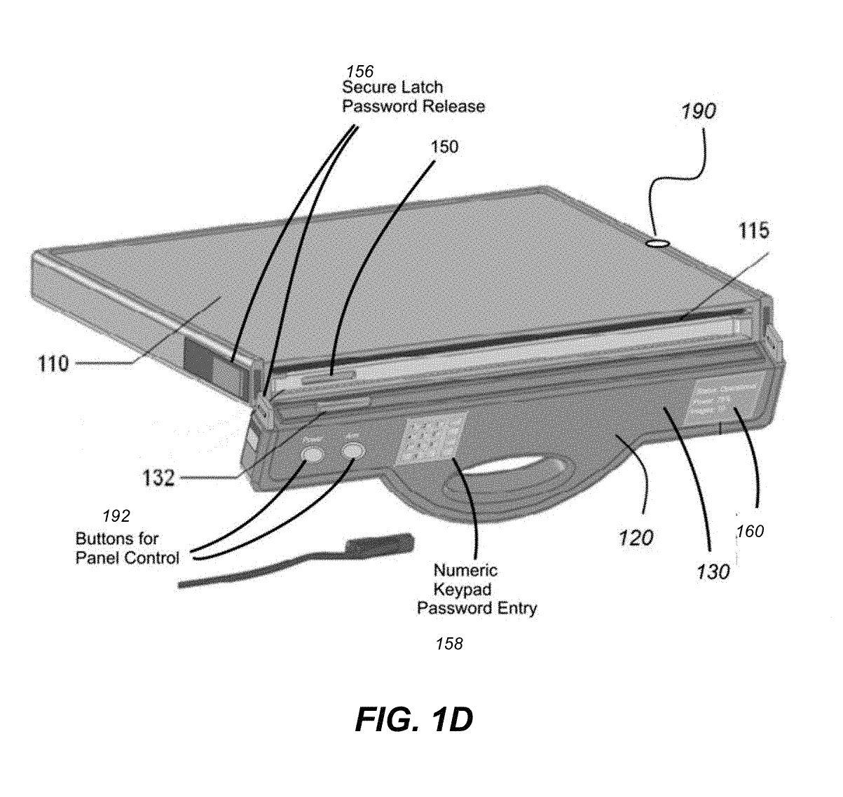

FIGS. 1A-1D are perspective diagrams that show an exemplary embodiment of a modular accessory enclosure 100, which may be referred to herein as a housing or as a shell, for a portable DR detector 150 according to one embodiment. The portable DR detector 150 has a back surface, visible in FIG. 1A, which may be referred to herein as a bottom surface. A top surface of the DR detector 150, not visible in FIG. 1A, faces in the opposite direction as the bottom surface. The top and bottom surfaces may be referred to herein as the major surfaces of the DR detector 150. Four side wall surfaces of the DR detector 150 extend between the edges of the major surfaces of the DR detector 150. As shown in FIG. 1A, modular accessory enclosure 100 for the portable DR detector 150 may include an environmental shell or housing 110 that provides an interior cavity to receive the DR detector 150. The housing 110 includes major top and bottom sections, or sides, disposed parallel to the top and bottom surfaces of the DR detector 150 when the DR detector 150 is inserted therein. The modular accessory enclosure 100 can selectively include a plurality of accessory functions, described herein, selected according to use, such as for a specific imaging application including, but not limited to, general radiography, CBCT (cone beam computed tomography), fluoroscopy or for field environment and radiographic imaging requirements. Environmental shell 110 may be a fluid-tight and particle-resistant enclosure and can be sealable, closable, or have one or more open sides. Shell 110 can be waterproof in its sealed position about the DR detector. Shell 110 can include a handle 130 that is integrally formed as a hinged member used to close an access opening of the shell 110, or the handle 130 may be otherwise attachable to the shell 110. Handle 130 can be formed to have an accessory cavity 120 therein for placement and storage of accessory components, as described herein. Shell 110 can be radiolucent entirely or only the top side of the shell 110 corresponding to the top surface of the DR detector 150 may be made from a radiolucent material, to allow radiographic imaging while enclosing the DR detector 150 therein. Shell 110 can have one or more radiopaque markers 190 that are visible in radiographic images captured by the enclosed DR detector 150 that may be used for orientation purposes or for geometric calibration, for example.

Shell 110 can have an electrical connector 132, used for establishing an electrical and mechanical connection to a power source of the DR detector 150 and/or to a processor or digital electronics of the DR detector 150. Shell 110 can provide an externally exposed connector 132′ to electrically and mechanically engage devices external to the DR detector to establish electrical and digital communication to the processor or digital electronics of the DR detector 150 via the electrical connector 132. Shell 110 can include one or more recesses, mounts, or pockets such as pocket 134 for receiving a radiopaque sheet, or layer, 115 such as a lead sheet to provide additional backscatter shielding. The radiopaque sheet 115 may also be made from a material designed for temperature control. The shell 110 may include bumpers 136 on side walls thereof for absorbing shock. The side walls of the shell 110 are disposed parallel to the side wall surfaces of the DR detector 150 when the DR detector 150 is inserted therein.

In one embodiment, a temperature control unit may be mounted within the modular accessory enclosure 100, touching or adjacent to one or more surfaces of the DR detector 150. Further, the temperature control unit can be configured to cool or heat a nearby surface of the DR detector from a single position. The temperature control unit can be responsive to a control signal for controlling a temperature magnitude. In one embodiment, the temperature control unit can maintain a selected temperature or temperature range for the inserted DR detector, wherein the range can be dependent on a detector operating mode. For example, a first temperature range can be selected for a dual energy operating detector mode. A second, smaller temperature range can then be selected for a tomosysnthesis operating detector mode. In one embodiment, the temperature control unit can be passive (e.g., an insulating sheet or sleeve). The temperature control unit can be used at a patient's bed, self-powered, or powered by an enclosed DR detector.

As shown in FIG. 1D, features of shell 110 can be modified for field operations, such as for veterinary or NDT applications. Features can include a secure latching device 156 that locks in a closed position a hinged handle 120. The latching device 156 may be used to prevent unwanted opening of the shell 110 via the hinged handle 120 and may be responsive to a predetermined password that is entered on a numeric keypad 158 to allow opening of the shell 110 via the hinged handle 120. A touch panel display 160 may allow input/output communication with the shell 110 or with the DR detector 150. Buttons 192 can be provided for panel control, such as to set up the DR detector for x-ray beam sensing and image capture. Shell 110 may include electronic memory to store images captured by DR detector 150 for subsequent downloading.

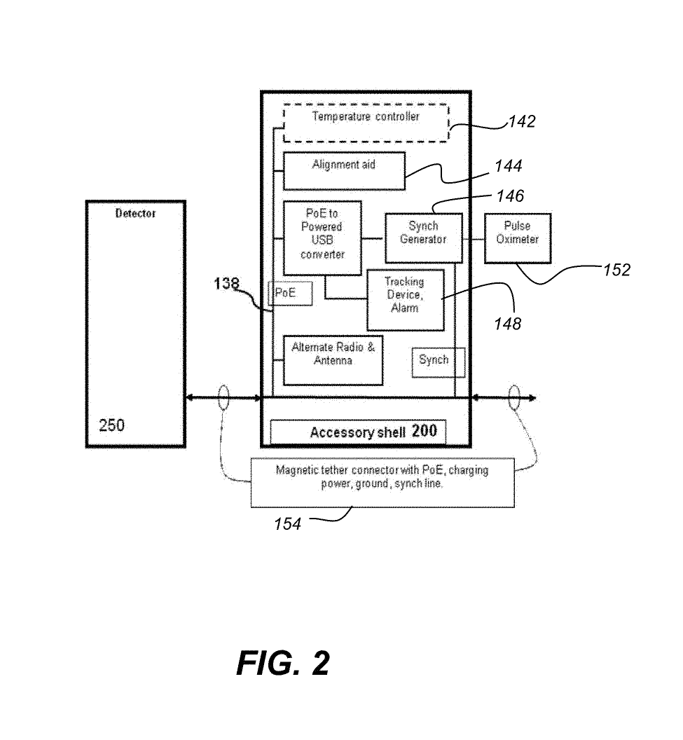

FIG. 2 is a schematic diagram of one embodiment of a modular accessory shell 200 for a portable DR detector 250. As shown in FIG. 2, a selectable set of accessories can be operatively coupled to a power/communication bus 138, or network, of a modular accessory shell 200. In one embodiment, the power/communication bus 138 can provide PoE (Power over Ethernet) to additional radio and/or other accessories. As shown in FIG. 2, accessories housed within modular accessory shell 200 can include a temperature controller 142, alignment aids 144, such as for tube and grid alignment or for source-to-detector alignment, additional wireless communication systems such as PoE connected radio such as UWB or alternative radio communication standards for mobile devices, additional power converters, USB or PoE powered accessories such as pulse oximeter 152, e.g., patient movement sensor, including synch generator 146, tracking/position locators 148 with an alternative audio alarms for unintended out-of-range transport or anti-theft warning. USB connection can be provided from the PoE to a USB converter. An optional magnetic tether connector 154 can be used for electrically linking the modular accessory shell 200 with the portable DR detector 250.

In one embodiment, a synch generator unit 146 can be mounted to an attachable DR detector 250 via the modular accessory container 200 to match x-ray activation timing with involuntary patient cardiac or respiratory movement detected by the pulse oximeter 152, and configured to communicate movement information to a controller of an associated radiographic imaging system through the attachable DR detector. The DR detector can use wired or wireless communication to transfer the movement information to the synch generator unit 146 which analyzes movement data, e.g., heartbeat, breathing, physical movement, in order to determine when the patient movement is at a constant relative position, e.g., at rest between heartbeats, breath held/released, and thereby provide a signal that may be used to activate x-ray exposure, or for multiple exposures, e.g., dual energy exposure.

In one embodiment, a tracking device 148 mounted to the modular accessory container 200 can be configured to emit wireless signals that report the current DR detector location when connected to a power source of an enclosed DR detector. In one embodiment, an associated radiographic imaging system can include additional hardware for monitoring a plurality of remote DR detectors to track their locations using corresponding tracking devices. In one embodiment, a central location can monitor a plurality of remote DR detectors for their location using corresponding powered tracking devices.

In one embodiment, accessories for the modular accessory shell 200 can use power supplied from a battery or power source of the DR detector 250 rather than requiring an additional battery in each of the accessory devices. As shown in FIG. 2, a user may simply recharge or replace the DR detector 250 battery rather than dealing with two or more separate energy sources. Alternatively, one or more additional batteries can be located in the accessory shell 200 rather than the DR detector 250, or in both to improve total energy capacity. The battery (or batteries) can be recharged through a single tether connection, or through a non-contact charge circuit that couples to the DR detector or accessory shell.

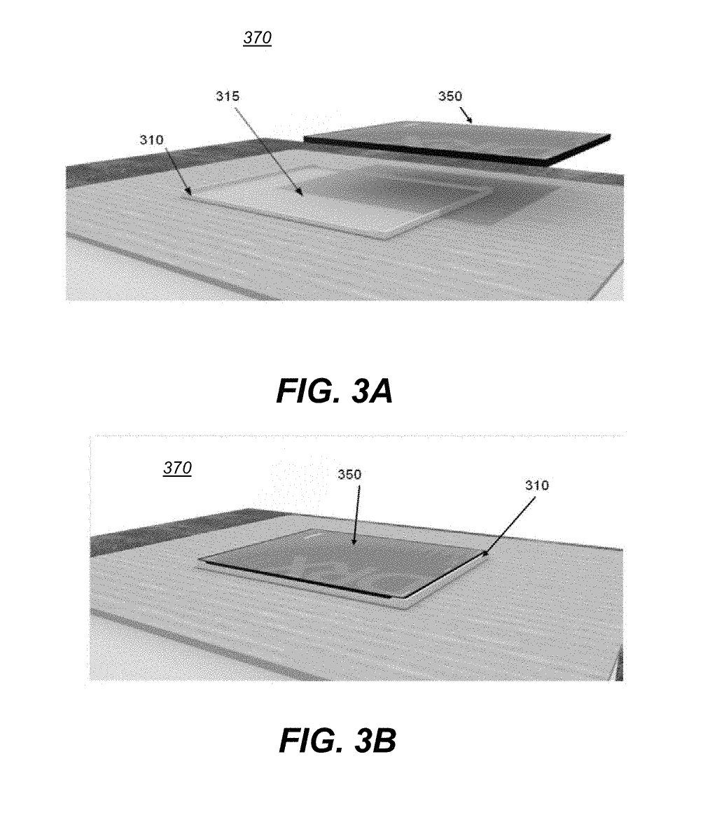

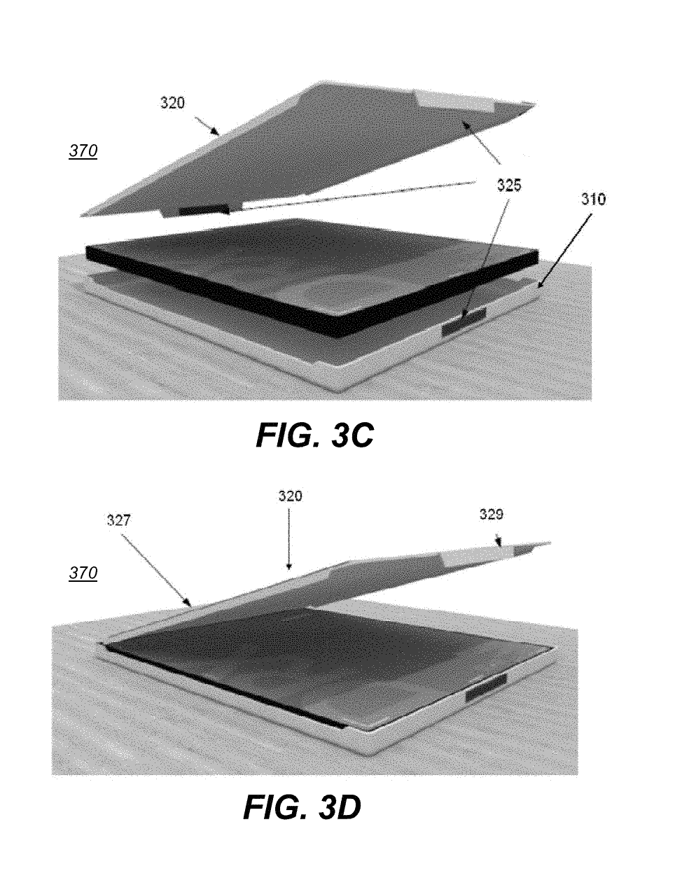

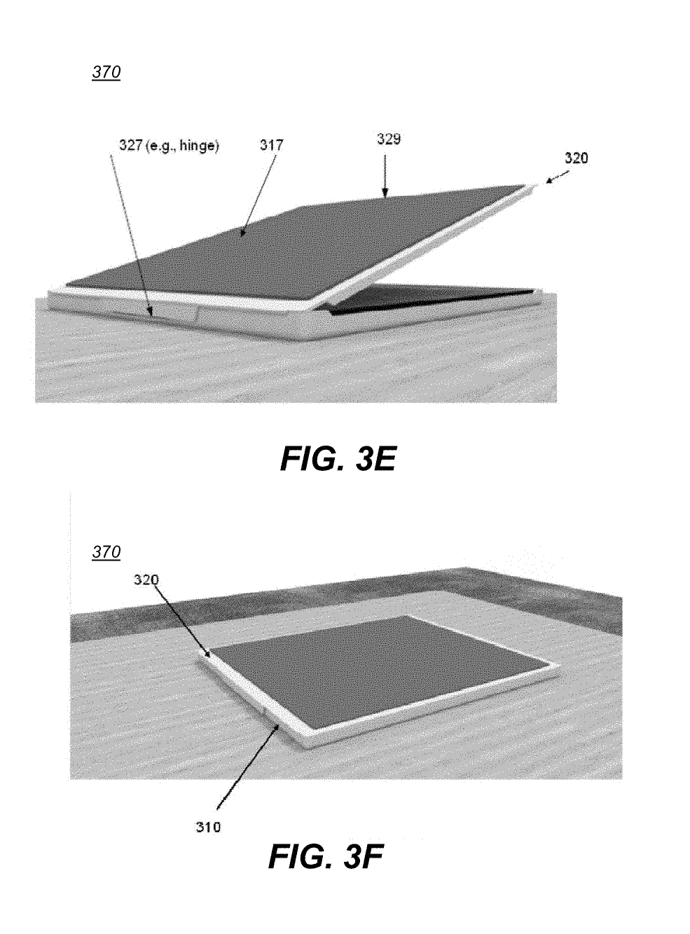

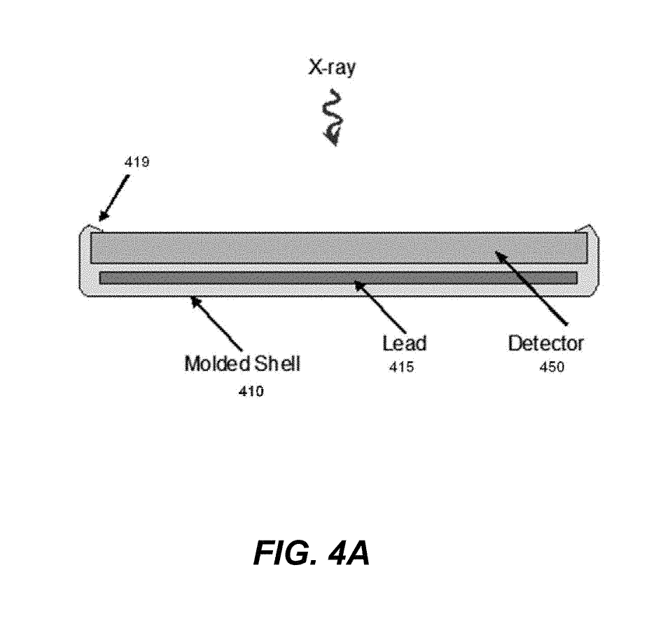

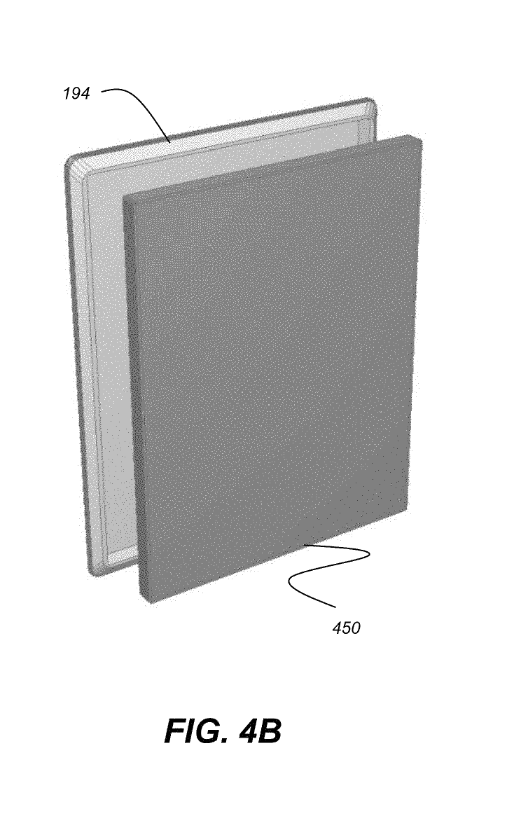

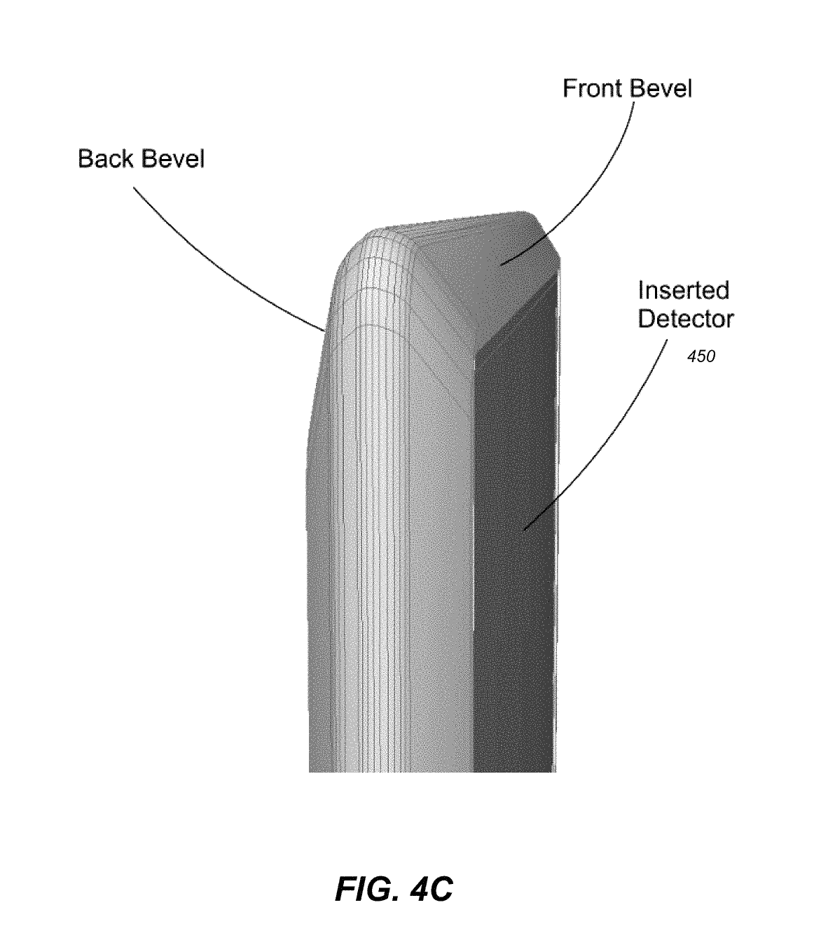

In one embodiment, as shown in FIGS. 3A-3F and 4A-4C, a low-cost foam cast accessory shell 310 can provide, for a modular accessory enclosure 370, a means to attach, encase, or retain an anti-scatter material or a sheet 315 of anti-scatter lead plating to a back of a DR detector 350. FIG. 3A is a perspective diagram that shows an exemplary embodiment of a modular accessory enclosure 370 for a portable DR detector according to an embodiment of the present disclosure. As shown in FIG. 3A, an accessory shell 310 with an integral, attached, or otherwise coupled anti-scatter plate 315 can be quickly and readily fitted to enclose a portable DR detector 350 without requiring difficult alignment or latching devices. The accessory shell 310 likewise can be quickly and easily removed from the DR detector 350 as shown in FIGS. 3A and 3B using, for example, felt pads that provide increased friction between the shell 310 (e.g., plastic) and the DR detector 350. This allows the back accessory shell 310 to be quickly removed, for example, to allow access to the back of the DR detector 350 housing, such as when the DR detector battery (not shown) needs to be replaced.

It is also be beneficial to use the accessory shell 310 without anti-scatter material to protect the back surface of the portable DR detector from scratches and abrasion. In one embodiment, the accessory shell can be a vacuum formed plastic shell. Vacuum-formed plastic material can be made very thin, e.g., on the order of 15 to 20 mils, so that the plastic accessory shell does not appreciably add to the overall thickness of the DR detector 350, and accordingly, the accessory shell 310 can be used with the portable DR detector 350 in standard radiological equipment such as in Bucky drawers without interference.

In one embodiment, the back cover accessory shell 310 may be placed without anti-scatter plate 315 over the (e.g., top and/or bottom) portable DR detector 350 to protect the top and/or bottom surface of the DR detector. Further, since the accessory plastic shell 310 is very low cost, the accessory shell 310 can be disposed of when worn from use and can be easily replaced with a new accessory shell 310 of the same type. In one embodiment, another complementary low cost accessory shell can be used to cover the top surface. As shown in FIG. 3C, a top cover accessory shell 320 in conjunction with the back shell can enclose and/or protect a portion of, or the entire portable DR detector 350 from surface abrasion and damage to due normal wear and tear. The top cover accessory shell 320 can be removably fastened and held to the back surface accessory shell 310. In one embodiment, the shell 370 may be formed from more than two connectable pieces. In one embodiment, fasteners 325 can securely, but removably affix the top cover accessory shell 320 to the accessory shell 310. Fasteners 325, including hook-and-loop fasteners (e.g., Velcro® fasteners or Velcro strips) can allow quick attachment and removal of the top accessory shell 320 to the bottom shell 310.

In one embodiment, the top accessory shell 320 can have a flexible flap 327 at one end of the shell as shown in FIGS. 3C and 3D. The flexible flap 327 can serve as a hinge for the top cover shell allowing for quick and easy insertion of the portable DR detector into the top and bottom accessory shells. In one embodiment, an opposing end of the shell 320 can have a flexible flap 329. Such an exemplary flexible flap arrangement can also be used to keep the top shell 320 and the bottom shell 310 connected together as a unit when not used.

In one embodiment, an accessory shell can also include an anti-scatter grid. As shown in FIG. 3E, an anti-scatter grid 317 can be attached to an outer surface of the accessory shell 320. The grid 317 can be securely but removably fixed to the accessory shell 320 (e.g., with a low tack force double side pressure sensitive adhesive (PSA)). Such an attachment can serve to hold the anti-scatter grid 317 firmly to the accessory shell 320 and because the adhesive has a low tack force, the grid 317 can be easily separated from an accessory shell such as when the accessory shell 320 becomes unusable due to excessive wear.

FIG. 3F is a diagram that depicts the portable DR detector 350 enclosed in a top and bottom accessory shell pair 310, 320 with an anti-scatter grid 317 attached to the outer surface thereof.

The versatility of a wireless portable DR detector includes its ability to be carried from one location to another throughout a medical facility. A portable DR detector is typically carried around by medical staff members who use the DR detector to image patients in different locations. Due to frequent handling there is an increased risk that an expensive portable DR detector might be dropped and damaged. One way to reduce this risk is to provide a non slip surface treatment to the outer surfaces of a DR detector. However, this is usually impractical because non slip surfaces can interfere with the placement of the DR detector under a patient. Also, a non slip surface over time will tend to wear out during normal usage and the non slip performance will degraded.

To reduce the risk of dropping a portable DR detector, medical staff can insert the portable DR detector into an accessory shell treated with a non-slip surface material that increases surface friction. When it is desired to place the portable DR detector under a patient the non slip accessory shell can be removed. Once the radiological images are taken the accessory shell can be placed back on the DR detector for carrying to another location. Since the accessory shell is made inexpensively it can be disposed of and replaced with a new one when the non slip surface treatment becomes degraded.

In one exemplary embodiment, it is advantageous to have an accessory shell that has had a non-slip surface treatment applied to the accessory shell. The accessory shell can appear as shown in FIG. 3B and could likewise be made of a low cost vacuum formed plastic with a non-slip surface treatment applied to its outer surfaces.

FIG. 4A is a diagram of an embodiment of a modular accessory container for a portable DR detector. As shown in FIG. 4A, in one embodiment, an accessory shell, or an accessory sleeve, 410 can be an open-sided shell, or a back cover shell (e.g., a one-sided shell can include or, as shown in FIG. 4A, exclude a backscatter shield 415 or anti-scatter grid). There are applications where a portable DR detector 450 may use only an open-sided shell 410, or back cover shell, where a top cover for the DR detector 450 is omitted because of added weight and x-ray absorption. As shown in FIG. 4A, in one embodiment, a seal or lip 419 can be formed on an open-sided shell that can be elastic secure the DR detector 450 in place, and/or to provide a liquid seal against a DR detector 450 having a carbon fiber top surface or cover.

In one embodiment, a handle 130 can be included as part of a detachable accessory shell or back cover accessory shell. FIG. 4B is an exploded view of a protective cast foam shell 194 that can be used with DR detector 450. The shell 194 may have rounded, inwardly sloped edges that provide low friction to facilitate DR detector 450 placement by sliding it beneath a bedridden patient. FIG. 4C show a perspective view of back and front bevels for DR detector 450.

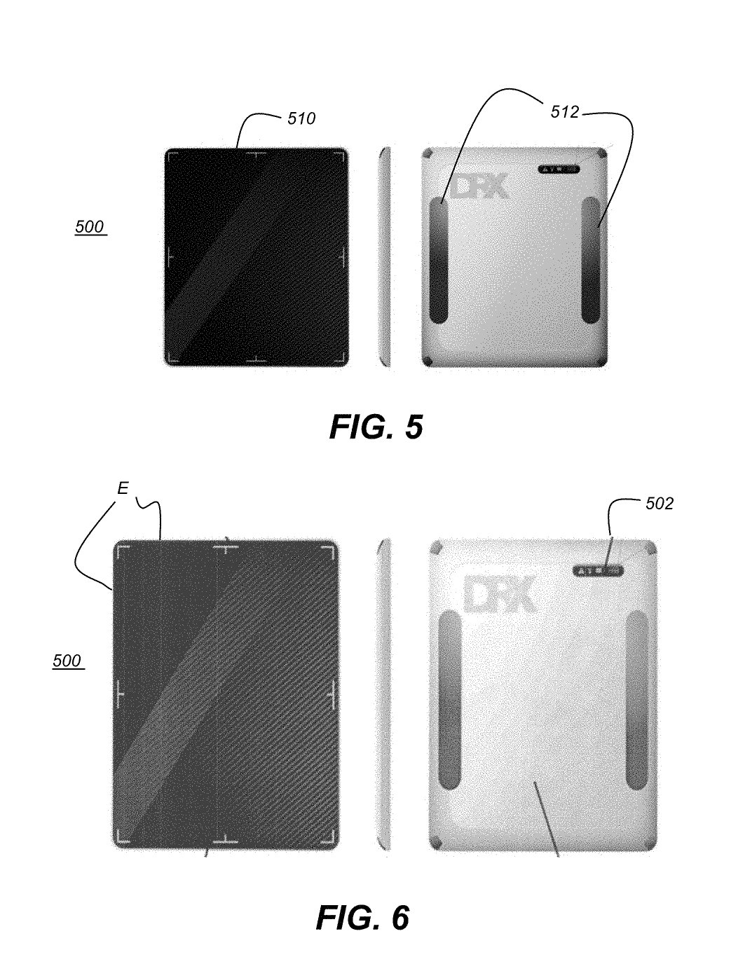

FIGS. 5 and 6 show top, side, and bottom views of a DR detector 500 that can be used with the modular accessory enclosure disclosed herein. DR detector 500 may be fabricated with a digital radiographic image sensor panel that is formed on a flexible substrate. The sensor panel can include an amorphous silicon (a-Si) sensor panel. The DR detector 500 can have a wireless or wired data or power interface. A protective shell 510 can provide indicators, sloped and rounded edges, and high friction hand grip regions 512. A low friction edge surround treatment as shown in FIG. 6 can help to provide shell 510 handling.

DR detector 500 has compact electronics packaging and a durable waterproof housing with low-attenuation edges. Edges E can be narrowed to lower attenuation along the periphery. Rounded edges E can be provided for ease of handling and patient comfort. A display 502 can show charge level and DR detector status, including status of image storage. Wireless or contact charge input can be provided. Wireless communication can also be provided for synchronization and image transfer.

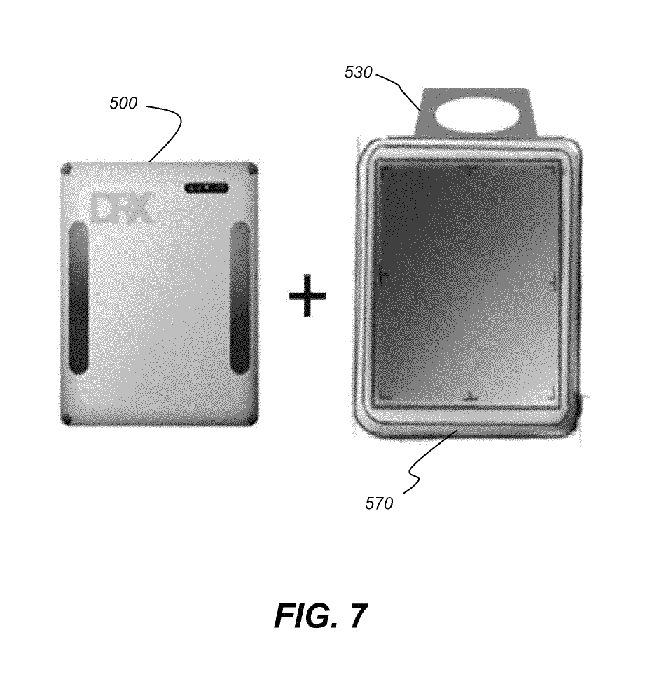

FIG. 7 shows a DR detector 500 along with a modular accessory enclosure 570 that is optimized for mobile imaging and can provide enhanced load-bearing capability and durability, while being very light weight and portable and allowing enhanced patient comfort. Enclosure 570 can provide a detachable grid and/or backscatter shield as described herein. Enclosure 570 can alternately provide an auxiliary energy source. A handle 530 can be removable. Enclosure 570 can support a number of features, including in-bin charging of the DR detector, so that no battery handling is required, low weight, and additional backscatter shielding.

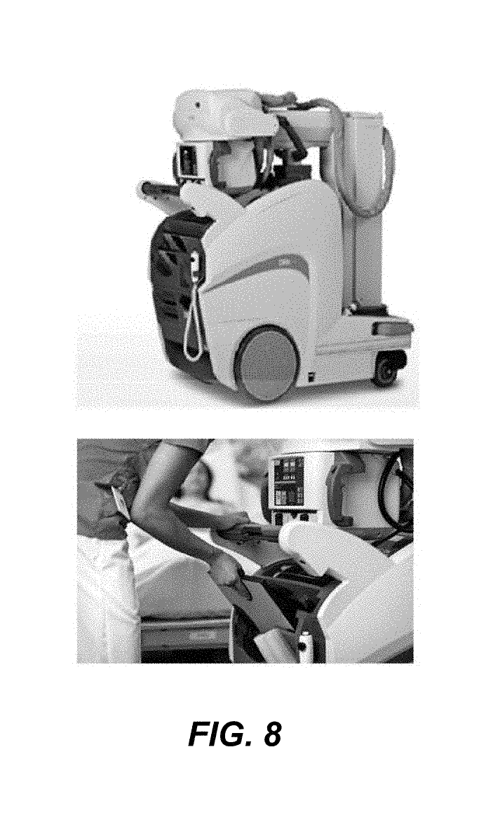

FIG. 8 is a perspective view that shows a mobile radiography apparatus that can use the DR detector disclosed herein provided with a modular accessory enclosure according to one embodiment. The lower portion of FIG. 8 shows in-bin charging during idle time or when transporting the DR detector on the mobile radiography apparatus.

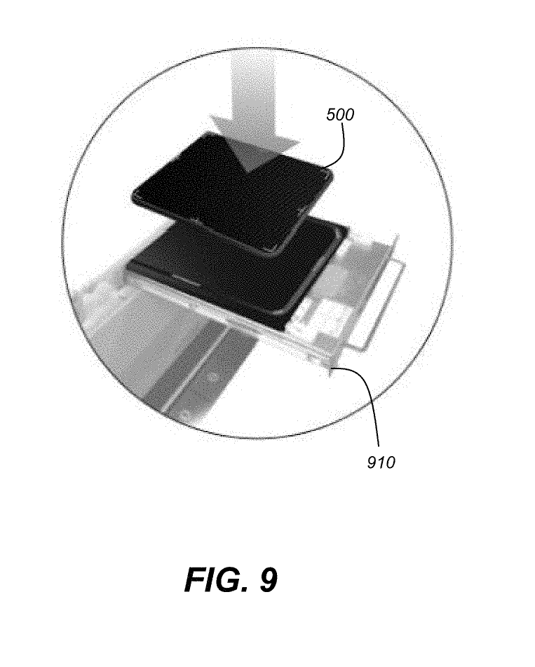

FIG. 9 shows use of DR detector 500 within a cassette frame accessory 910. This arrangement provides an ISO compliant geometry for use with existing bucky equipment. This arrangement also provides an adapter for an existing tether connector and a snap-on adapter for the DR detector as well as an optional auxiliary battery.

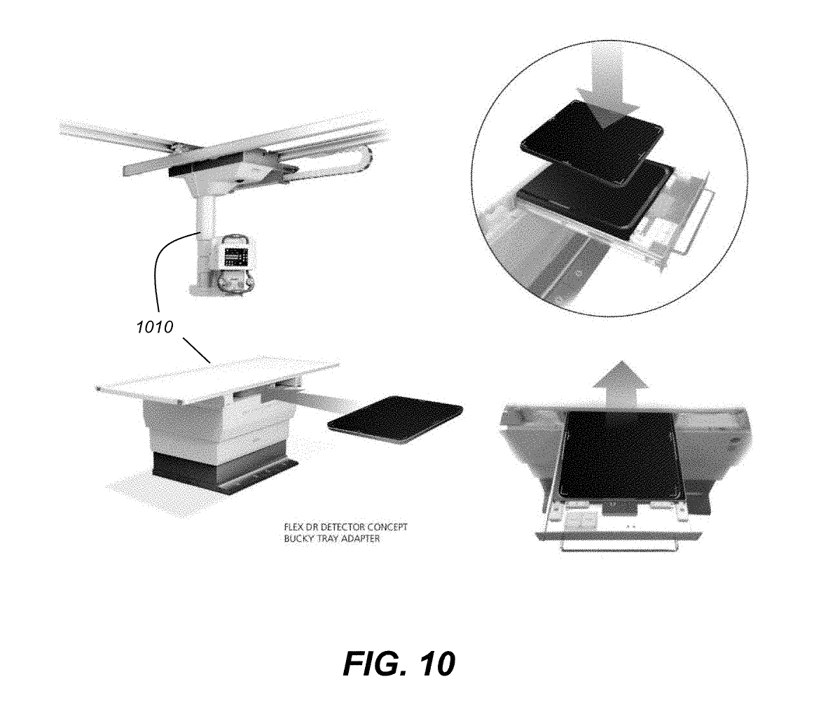

FIG. 10 shows a DR detector 500 used with a bucky adapter 1010 according to an embodiment of the present disclosure. This arrangement features an ISO film cassette size shell with an auxiliary energy source and tether interface, using a light-weight, durable portable rigid DR detector 500.

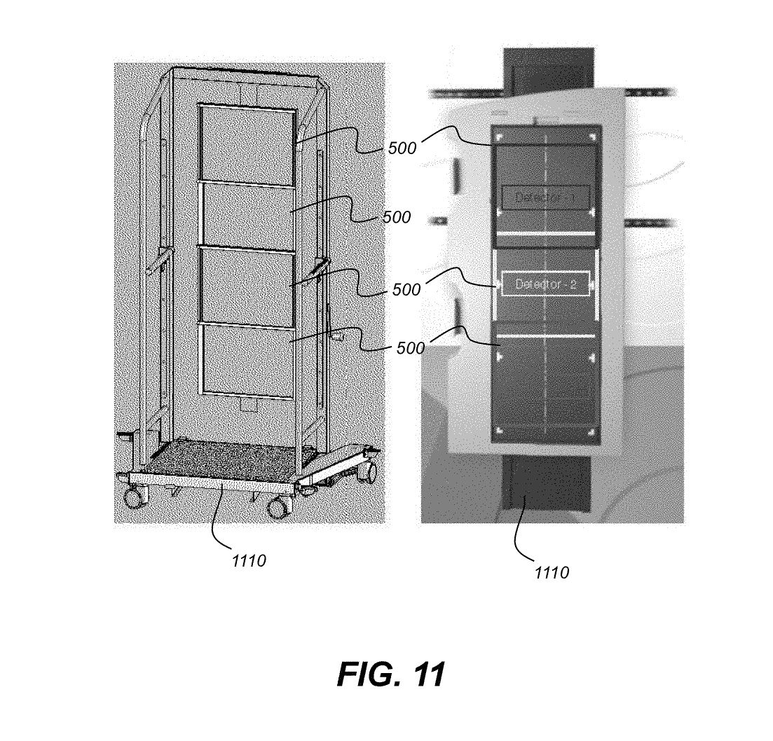

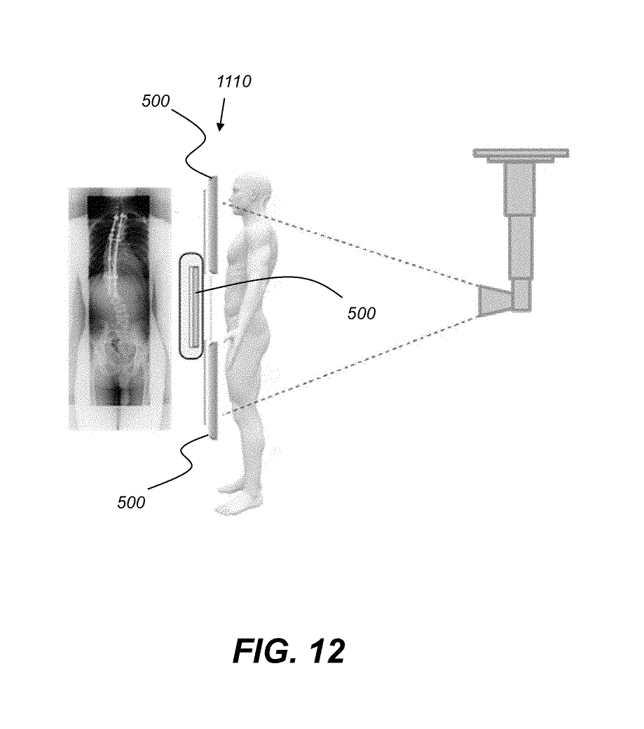

FIGS. 11 and 12 show an embodiment using a long-length imaging stand 1110 for cassette positioning. Imaging stand 1110 can hold a number of DR detectors, such as three or four DR detectors 500 as shown. As shown on the right hand side of FIG. 11, top and bottom DR detectors 500 can be stationary, with the middle DR detector 500 being movable. Calibration and stitching algorithms and multi-detector synchronization can be used with this arrangement. This configuration can be advantageous for extended field tomosynthesis with successive single-shot capture, providing reduced patient motion artifacts. A low attenuation DR detector edge design can reduce image artifacts at the seams between images.

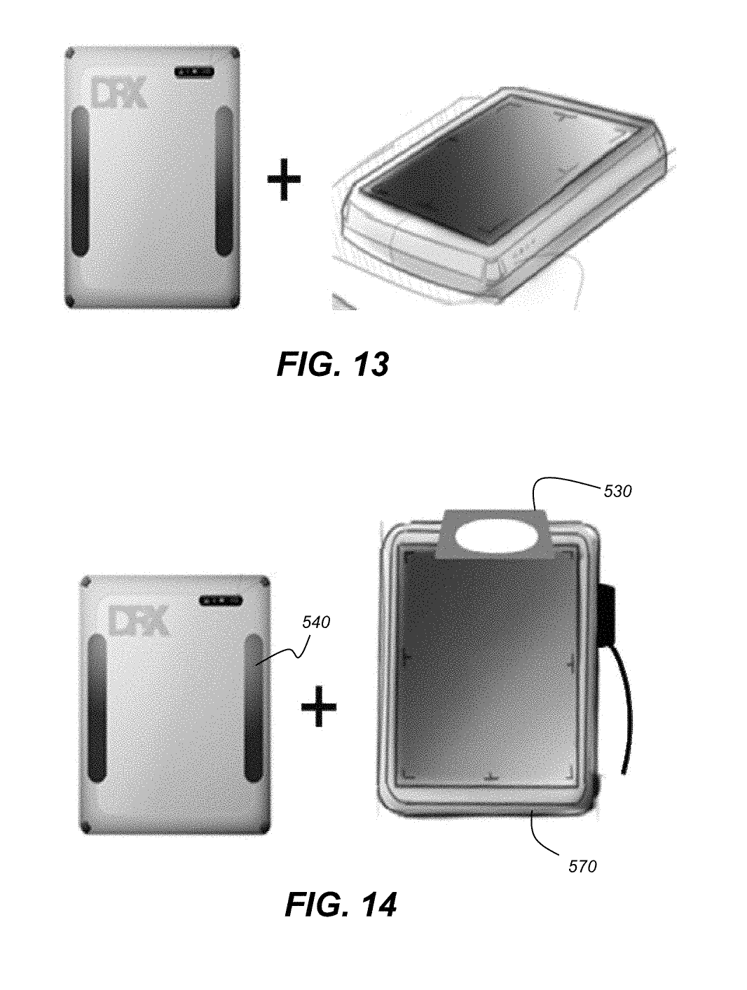

FIG. 13 shows an embodiment with enhanced DR detector source position orientation detection for improved geometric calibration. Internal position sensors can be provided. This configuration can use patient cardiac/respiratory monitoring for optimal x-ray pulse synchronization.

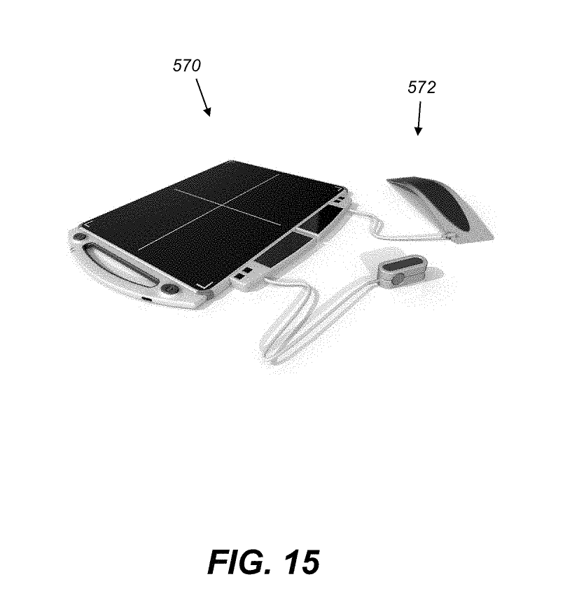

FIG. 14 shows an embodiment of modular accessory enclosure 570 suited for bedside imaging wherein handle 530 and one or more gripping pads 540 are provided. The embodiment shown can provide imaging at up to 10 frames per second using a mobile radiography apparatus as shown in FIG. 8. An optical tether connection can be provided for real-time display. FIG. 15 shows an embodiment of a modular accessory enclosure 570 with respiratory and pulse oximeter monitors 572.

Exemplary embodiments herein can be applied to digital radiographic imaging panels that use indirect DR detectors having a scintillating screen and/or direct DR detectors having an array of pixels comprising X-ray absorbing photoconductors and readout circuits.

It should be noted that while the present description and examples are primarily directed to radiographic medical imaging of a human or other subject, embodiments of apparatus and methods of the present application can also be applied to other radiographic imaging applications. This includes applications such as non-destructive testing (NDT), for which radiographic images may be obtained and provided with different processing treatments in order to accentuate different features of the imaged subject.

Embodiments of radiographic imaging systems and/methods described herein contemplate methods and program products on any computer readable media for accomplishing its operations. Certain exemplary embodiments accordingly can be implemented using an existing computer processor, or by a special purpose computer processor incorporated for this or another purpose or by a hardwired system.

Consistent with exemplary embodiments, a computer program with stored instructions that perform on image data accessed from an electronic memory can be used. As can be appreciated by those skilled in the image processing arts, a computer program implementing embodiments herein can be utilized by a suitable, general-purpose computer system, such as a personal computer or workstation. However, many other types of computer systems can be used to execute computer programs implementing embodiments, including networked processors. Computer program for performing method embodiments or apparatus embodiments may be stored in various known computer readable storage medium (e.g., disc, tape, solid state electronic storage devices or any other physical device or medium employed to store a computer program), which can be directly or indirectly connected to the image processor by way of the internet or other communication medium. Those skilled in the art will readily recognize that the equivalent of such a computer program product may also be constructed in hardware. Computer-accessible storage or memory can be volatile, non-volatile, or a hybrid combination of volatile and non-volatile types.

It will be understood that computer program products implementing embodiments of this application may make use of various image manipulation algorithms and processes that are well known. It will be further understood that computer program products implementing embodiments of this application may embody algorithms and processes not specifically shown or described herein that are useful for implementation. Such algorithms and processes may include conventional utilities that are within the ordinary skill of the image processing arts. Additional aspects of such algorithms and systems, and hardware and/or software for producing and otherwise processing the images or co-operating with computer program product implementing embodiments of this application, are not specifically shown or described herein and may be selected from such algorithms, systems, hardware, components and elements known in the art.

While the invention has been illustrated with respect to one or more implementations, alterations and/or modifications can be made to the illustrated examples without departing from the spirit and scope of the appended claims. In addition, while a particular feature of the invention can have been disclosed with respect to only one of several implementations/embodiments, such feature can be combined with one or more other features of the other implementations/embodiments as can be desired and advantageous for any given or particular function.

What is claimed is:

1. An enclosure for a digital radiography detector, the enclosure comprising:
a housing that is configured to enclose the detector;
a handle attached to the housing;
a connector in the housing that is configured to electrically connect to the detector and an external tether; and
an accessory cavity in a portion of the enclosure to provide storage space for an accessory that is electrically connectable to the detector.

2. The enclosure of claim 1, wherein the accessory comprises a synch generator configured to detect patient movement control x-ray activation timing with respect to the patient movement.

3. The enclosure of claim 1, wherein the accessory comprises a tracking device configured to communicate data that identifies a current location of the enclosed DR detector.

4. The enclosure of claim 1, wherein the accessory is electrically coupled to a power source for the DR detector.

5. The enclosure of claim 1, wherein the housing comprises a top and a bottom section each corresponding to a major top and bottom surface of the detector, respectively.

6. The enclosure of claim 1, wherein the housing comprises a hinged member to open and close an access opening used to insert the DR detector into the enclosure.

7. The enclosure of claim 1, wherein the housing is configured to fit into a bucky of a radiographic imaging system such that the enclosed detector remains operable to capture radiographic images in a normal course of usage of the radiographic imaging system.

8. The enclosure of claim 1, wherein the accessory cavity is formed inside the handle.

9. The enclosure of claim 1, further comprising an anti-scatter grid that is removably attached to the housing.

10. The enclosure of claim 9, further comprising an adhesive to attach the grid to the housing.

11. The enclosure of claim 9, further comprising a sleeve in the housing to secure the grid.

12. The enclosure of claim 1, further comprising a material on an exterior surface of the housing having a coefficient of static friction greater than a coefficient of static friction of a surface of the DR detector.

13. The enclosure of claim 6, wherein the housing is configured to be waterproof when the access opening is closed by the hinged member.

14. The enclosure of claim 1, wherein the housing comprises a radiolucent side corresponding to a top surface of the detector to allow radiographic image capture therein.

15. The enclosure of claim 1, further comprising a radiopaque marker in or on the housing, the radiopaque marker configured to be visible in a radiographic image captured by the enclosed DR detector.

16. The enclosure of claim 6, further comprising a latching device configured to lock the hinged member in a closed position, the latching device further configured to receive a predetermined password code causing the latching device to unlock the hinged member.

17. The enclosure of claim 1, further comprising a touch sensitive display for digital input/output communication with the DR detector and the housing.

18. A method comprising:
providing a shell having an interior space configured to receive and enclose a DR detector and having a cavity to receive an accessory;
inserting the DR detector into the shell;
electrically and mechanically connecting a tether to the enclosed detector through an opening in the shell;
placing a position locator within the cavity, the position locator configured to determine a location of the position locator and to transmit location data that identifies the location of the position locator; and
transmitting a radiographic image captured by the enclosed DR detector over the tether.

19. The method of claim 18, further comprising sealing the shell in a fluid tight fashion after the step of inserting the DR detector into the shell.

20. The method of claim 18, further comprising providing a portable power source in the DR detector for powering the DR detector, and providing power to an accessory in the cavity using the portable power source of the DR detector.
